(19) United States
(12) Patent Application Publication
(10) Pub. No.: US 2002/0091436 A1
(43) Pub. Date: Jul. 11, 2002

(54) Cover stent for the treatment of vulnerable atherosclerosis plaque

(76) Inventors: David Y. Phelps, Anchorage, KY (US); Gregory Furnish, Louisville, KY (US)

Correspondence Address: Donald N. Halgren, 35 Central Street, Manchester, MA, 01944, US

(21) Appl. No.: 10/098,133

(22) Filed: Mar. 13, 2002

Related U.S. Application Data
(63) Continuation of application No. 09/499,190, filed on Feb. 7, 2000, now abandoned.

(51) Int. Cl. A61F 2/06

(52) U.S. Cl. 623/1.11

(57) ABSTRACT

The present invention comprises a stent barrier assembly deliverable into a coronary artery for the segregating and sheilding of the vulnerable plaque in the artery wall from the bloodstream, comprising a compressible stent arranged on the distal end of a catheter. A fluid-impermeable, compressible stent cover is arranged about the stent to provide a barrier to plaque and lipid pool within the artery.

### BACKGROUND OF THE INVENTION

1. Field of the Invention

This invention relates to treatment of arterial disease and more particularly to the use of a stent in an artery for treatment of that disease.

2. Prior Art

Heart disease is the leading killer of men and women in the world today. Heart disease in many instances may be characterized as coronary artery disease. When the coronary arteries become blocked, symptoms ranging from angina to heart attacks, may occur. In a percentage of these cases, the coronary arteries may be unblocked through a non invasive technique such as balloon angioplasty. Some five hundred to six hundred thousand angioplasties are performed each year within the United States. Where balloon angioplasty may not be appropriate, a bypass of the occluded or blocked vessel may be necessary. Identifying an opening such occlusions is known to give relief to the symtoms of angina, but it is also known that they do little to prolong life expectancy. The real killer in this coronary artery disease is often sudden blockages that are caused not by the slow accumulation of plaque that gradually block off the arteries, but by a sudden thrombosis (clotting) of the arteries caused by what are now referred to as “vulnerable plaque”.

Vulnerable plaques are defined as plaques prone, in the presence of an appropriate trigger, to events such as ulceration rupture, erosion or thrombus that can lead to an acute syndrome. Those events are believed to share three common characteristics, a large lipid pool, a thin fibrous tab and macrophage infiltration.

Current methods of diagnosing arterial disease, using such as stress tests as angiograms, are inadequate at detecting these “vulnerable plaques”. Therefore, in most instances, this potentially lethal condition often goes untreated.

It is an object of the present invention, to provide treatment options for this particular condition.

It is a further object of the present invention to provide a plaque treatment utilizing an improved stent apparatus.

### BRIEF SUMMARY OF THE INVENTION

The present invention comprises a covered stent arrangement so as to reinforce and contain the arterial wall at the site of the vulnerable plaque. The covered “stent” would be utilized to support the fibrous cap and isolate the lipid pool from the bloodstream traveling within the artery. The stent of the present invention may be made from stainless steel, nitinol or other bio compatible material and introduced into the artery on the distal end of a delivery catheter. The delivery catheter has an elongated guidewire extending therethrough. The catheter itself, is comprised of a polymeric material suitable for compression and delivery within an artery. The stent is in its compressed configuration adjacent the distal end of the catheter shaft. A stent sheath or cover also is in a compressed or collapsed configuration about the compressed stent during its delivery within the artery. The guidewire extends through the compressed stent and is used to deliver the stent to the site of the vulnerable plaque. Upon the delivery to the situs of the vulnerable plaque, the guidewire may be rotated and withdrawn from within the compressed sheath and stent while simultaneously withdrawing the catheter sheath therefrom to permit the stent and compressed sheath to expand and engage the artery wall.

The stent sheath or cover is preferably made from an elastomeric material, such as silicone, which will function as a blood-lipid barrier. The intent of the sheath is to effect a shield for the bloodstream, from the vulnerable plaque and lipid pool thereadjacent.

In a further embodiment of the present invention, the stent sheath may be multilayered, the innermost layer of the sheath acting as the liquid shield and barrier, the outer layer of the sheath acting as a delivery medium for curative medicaments or pharmaceutical agents to be delivered to the plaque over a period of time.

The invention thus comprises a stent barrier assembly deliverable into a coronary artery for the segregating and sheilding of the vulnerable plaque in the artery wall from the bloodstream, comprising: a compressible stent arranged on the distal end of a catheter; a fluid-impermeable, compressible stent cover arranged about the stent to provide a barrier to plaque and lipid pool within the artery. The compressible cover and the compressible stent are arranged compressed in a delivery catheter sheath. The stent cover may be comprised of a plurality of layers. One of the layers may be an outer layer comprised of a medicament delivery material. The outer layer may includes a curative medicament therein. One of the layers may be an inner layer. The inner layer may be fluid impermeable.

The invention also include a method of segregating and shielding the vulnerable plaque in an artery wall from the bloodstream, comprising the steps of: arranging a stent on the distal end of a delivery catheter; placing a cover around the stent in the delivery catheter; inserting the stent into the artery adjacent the vulnerable plaque;.and withdrawing the delivery catheter from the stent, so as to permit the stent and cover thereon to expand within the artery to provide a barrier between the vulnerable plaque and the bloodstream.

Thus what has been shown is a unique barrier and delivery device to safely enclose and provide a shield or barrier to vulnerable atherosclerosis, plaque. By segregating such vulnerable plaque from the bloodstream, the likelihood of a sudden thrombosis of the arteries is minimized.

### BRIEF DESCRIPTION OF THE DRAWINGS

The objects and advantages of the present invention will become more apparent, when viewed in conjunction with the following drawings in which:

FIG. 1 is a side elevational view of a catheter and guidewire arrangement according to the principles of the present invention;

FIG. 2 is a side elevational view, in section, of an artery with a stent with a sheath thereon arranged therewithin; and

FIG. 3 is a sectional view of a part of the stent sheath or cover.

### DESCRIPTION OF THE PREFERRED EMBODIMENTS

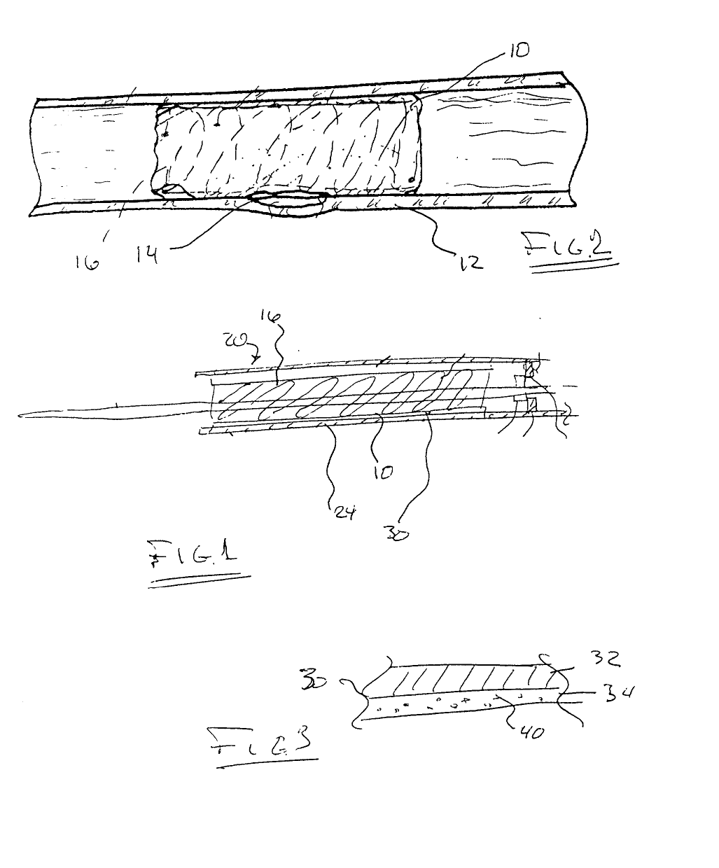

Referring now to the drawings in detail, and particularly to FIGS. 1 and 2, there is shown the present invention which comprises a covered stent arrangement 10 so as to reinforce and contain the arterial wall 12 at the site of the vulnerable plaque 14. The covered “stent” 10 would be utilized to support the fibrous cap and isolate the lipid pool, as shown in FIG. 1, from the bloodstream traveling within the artery 12. The stent structure 16 of the present invention may be made from stainless steel, nitinol or other bio compatible material and introduced into the artery on the distal end of a delivery catheter 20, as shown in FIG. 1. The delivery catheter 20 has an elongated guidewire 22 extending therethrough. The catheter 20 has an outer sheath 24, is comprised of a polymeric material suitable for compression and delivery within an artery 12. The stent structure 16 is in its compressed configuration adjacent the distal end of the catheter shaft 20. A stent sheath or cover 30 also is in a compressed or collapsed configuration about the compressed stent for insertion and its delivery within the artery 12. The guidewire 22 extends through the compressed stent 16 and is used to deliver the stent arrangement 10 to the site of the vulnerable plaque 14. Upon the delivery to the situs of the vulnerable plaque, the guidewire 22 may be rotated and withdrawn from within the compressed sheath 30 and stent 16 while simultaneously withdrawing the catheter sheath 24 therefrom to permit the stent 16 and compressed sheath or cover 30 to expand and engage the artery wall 30, and shield the vulnerable plaque 14.

The stent sheath or cover 30 is preferably made from an elastomeric material, such as silicone, which will function as a blood-lipid barrier. The intent of the sheath 30 is to effect a shield for the bloodstream, from the vulnerable plaque and lipid pool 30 thereadjacent.

In a further embodiment of the present invention, as shown in FIG. 3, the stent sheath 30 may be multilayered, having an innermost layer 32 of the sheath 30 acting as the liquid shield and barrier, and an outer layer 34 of the sheath 30 acting as a porous delivery medium for curative medicaments or pharmaceutical agents 40 contained therewithin, and to be delivered to the vulnerable plaque 14 over a period of time.

Thus what has been shown is a unique barrier and delivery device to safely enclose and provide a shield or barrier to vulnerable atherosclerotic plaque. By segregating such vulnerable plaque from the bloodstream, the likelihood of a sudden thrombosis of the arteries is minimized.

We claim:

1. A stent barrier assembly deliverable into a coronary artery for the segregating and sheilding of the, vulnerable plaque in the artery wall from the bloodstream, comprising:
a compressible stent arranged on the distal end of a catheter;
a fluid-impermeable, compressible stent cover arranged about said stent to provide a barrier to plaque and lipid pool within the artery.

2. The stent barrier assembly as recited in claim 1, wherein said compressible cover and said compressible stent are arranged compressed in a delivery catheter sheath.

3. The stent barrier assembly as recited in claim 1, wherein said stent cover is comprised of a plurality of layers.

4. The stent barrier assembly as recited in claim 3, wherein one of said layers is an outer layer comprised of a medicament delivery material.

5. The stent barrier assembly as recited in claim 4, wherein said outer layer includes a curative medicament therein.

6. The stent barrier assembly as recited in claim 3, wherein one of said, layers is an inner layer.

7. The stent barrier assembly as recited in claim 6, wherein said inner layer is fluid impermeable.

8. A method of segregating and shielding the vulnerable plaque in an artery wall from the bloodstream comprising the steps of:
arranging a stent on the distal end of a delivery catheter;
placing a cover around said stent in said delivery catheter;
inserting said stent into said artery adjacent said vulnerable plaque; and
withdrawing said delivery catheter from said stent, so as to permit said stent and cover thereon to expand within said artery to provide a barrier between the vulnerable plaque and the bloodstream.
